(19) United States
(12) Patent Application Publication
(10) Pub. No.: US 2019/0153488 A1
(43) Pub. Date: May 23, 2019

(54) INTEGRATED BIOLOGICAL CONVERSION OF GASEOUS SUBSTRATE INTO LIPIDS

(71) Applicant: Massachusetts Institute of Technology, Cambridge, MA (US)

(72) Inventors: Gregory Stephanopoulos, Winchester, MA (US); Peng Hu, Durham, NH (US); Hongjuan Liu, Quincy, MA (US)

(73) Assignee: Massachusetts Institute of Technology, Cambridge, MA (US)

(21) Appl. No.: 16/320,740

(22) PCT Filed: Sep. 6, 2016
(86) PCT No.: PCT/US2016/050369

§ 371 Date: Jan. 25, 2019

(51) Int. Cl. C12P 7/64; C12P 39/00

(52) U.S. Cl. CPC C12P 7/6409; C12P 39/00

(57) ABSTRACT

A bioconversion scheme is provided that effectively converts syngas, generated from gasification of coal, natural gas or biomass, into lipids that can be used for biodiesel production.

### RELATED APPLICATION

This application is a national stage filing under 35 U.S.C. § 371 of International Patent Application Serial No. PCT/US2016/050369, filed Sep. 6, 2016, the content of which is incorporated by reference herein in its entirety.

### GOVERNMENT SUPPORT

This invention was made with Government support under Grant No. DE-AR0000059 awarded by the Department of Energy (DOE). The Government has certain rights in the invention.

### FIELD OF THE DISCLOSURE

A bioconversion scheme is provided that effectively converts syngas, generated from gasification of coal, natural gas or biomass, into lipids that can be used for biodiesel production.

### BACKGROUND

Concerns over diminishing oil reserves and climate-changing greenhouse gas emissions have led to calls for clean and renewable liquid fuels (1). One promising direction has been the production of microbial oil from carbohydrate feedstocks. This oil can be readily converted to biodiesel and recently there has been significant progress in the engineering of oleaginous microbes for the production of lipids from sugars (2, 3, 4, 5). A major problem with this approach has been the relatively high sugar feedstock cost. Alternatively, less costly industrial gases containing CO₂ with reducing agents, such as CO or H₂, have been investigated. In one application, anaerobic Clostridia have been used to convert synthesis gas to ethanol (6), albeit at low concentration requiring high separation cost.

### SUMMARY

Disclosed herein is an alternative gas-to-lipids approach that overcomes the drawbacks of previous schemes. It has previously been shown that acetate in excess of 30 g/L can be produced from mixtures of CO₂ and CO/H₂ using an evolved strain of the acetogen M. thermoacetica, with a substantial productivity of 0.55 g/L/h and yield of 92% (7). It also has been demonstrated that the engineering of the oleaginous yeast Y. lipolytica can yield biocatalysts that can produce lipids from glucose at high yields and productivities (2, 5). At the same time, as part of the quest for inexpensive feedstock, lipid production from other substrates such as acetate and other volatile fatty acids (VFAs) that can be potentially sourced at lower costs as products of anaerobic digestion or from exhaust gases in steel manufacturing has been investigated.

As demonstrated herein, the above two systems can be integrated into a two-stage process whereby CO₂ and CO/H₂ are converted to acetic acid in the first stage anaerobic bubble column bioreactor and the product acetic acid is subsequently converted to lipids by Y. lipolytica in a second stage aerobic bioreactor. To assess the merits of this approach, fermentations of M. thermoacetica and Y. lipolytica were individually optimized with the intent to maximize acetate and lipid yield and productivity, respectively. A hollow fiber membrane filter was deployed in the anaerobic bioreactor to allow continuous removal of the acetic acid product and recycle of M. thermoacetica cells to the bubble anaerobic column. Similarly, cell recycle was also used in the second bioreactor in order to decouple the residence time required for growth and lipogenesis in Y. lipolytica cells from that of acetic acid consumption and therefore allow for the development of a dense microbial culture and high lipid concentration in the second bioreactor. For the first time, the separation of the growth phase of M. thermoacetica from the phase of acetic acid production was demonstrated: first a robust culture of M. thermoacetica is established in a CO-dependent growth phase by using a CO₂/CO gas mixture, and then the gas composition is switched to a H₂/CO₂ mixture that produces acetate at significantly higher specific productivity. This advance allowed securing of both high specific productivity and a high cell density of M. thermoacetica for an overall very high volumetric productivity of 0.9 g of acetate/L/h. The two processes were then integrated in a single continuous flow gas-to-oil processing scheme (FIG. 1), with design decisions based on the performance characteristics of the individual reactors.

Disclosed herein are methods of converting a gaseous substrate into a lipid. In some embodiments, the method comprises culturing a first organism in the presence of the gaseous substrate under conditions suitable for the first organism to reduce carbon dioxide in the gaseous substrate in the presence reducing agents, wherein the organism synthesizes one or more volatile fatty acids by reduction of the carbon dioxide. In some embodiments, the method comprises culturing a second organism in the presence of the volatile fatty acids produced by the first organism under conditions suitable for the second organism to convert the volatile fatty acids into a lipid. In some embodiments, the first and second processes are integrated so that they comprise a continuous process scheme in which the gaseous substrate is transformed to a lipid.

In some embodiments of the disclosed methods, the gaseous substrate comprises carbon dioxide (CO₂), and one or more of hydrogen (H₂) and/or carbon monoxide (CO). For example, in some embodiments, the gaseous substrate comprises a mixture of carbon monoxide and carbon dioxide, a mixture of hydrogen and carbon dioxide, or a mixture of hydrogen, carbon monoxide and carbon dioxide. In some embodiments, the carbon dioxide is used as the carbon source for the synthesis of the volatile fatty acid. In some embodiments, the hydrogen is used as an electron donor, serving to reduce the carbon dioxide. In some embodiments, the carbon monoxide is used as an electron donor serving to reduce the carbon dioxide. In some embodiments, the gaseous substrate comprises an industrially available gas mixture known as synthesis gas, also known as syngas.

In some embodiments, the volatile fatty acids produced comprise acetic acid. In some embodiments, the volatile fatty acids comprise methanoic acid and/or acetic acid and/or propionic acid and/or butyric acid and/or isobutyric acid and/or valeric acid, and/or isovaleric acid.

In some embodiments of the methods presently disclosed, the first organism is a prokaryote anaerobe capable of utilizing carbon dioxide as a carbon source. In some embodiments, the organisms utilized in the first bioreactor are selected from the group consisting of Moorella thermoacetica, Clostridium ljungdahlii, Clostridium carboxidivorans P7T, Clostridium ragsdalei, Alkalibaculum bacchi, C. autoethanogenum, Clostridium drakei, and/or Butyribacterium methylotrophicum.

In some embodiments, the first organism is capable of capturing carbon sourced from carbon dioxide as acetyl-CoA with a rate that is at least 1 g acetic acid/L-hr, such as at least 1.1 g acetic acid/L-hr, 1.2 g acetic acid/L-hr, 1.3 g acetic acid/L-hr, 1.4 g acetic acid/L-hr, 1.5 g acetic acid/L-hr, 1.6 g acetic acid/L-hr, 1.7 g acetic acid/L-hr, 1.8 g acetic acid/L-hr, 1.9 g acetic acid/L-hr, 2 g acetic acid/L-hr or 2.5 g acetic acid/L-hr; and/or an efficiency that is at least 92%, such as at least 93%, 94%, 95%, 96%, 97%, 98%, or 99%.

In some embodiments, the organism(s) utilized in the first (anaerobic) bioreactor are thermophiles, having an optimal growth temperature (Tₒₚₜ) that is greater than 40° C., or greater than 45° C., or greater than 50° C., or greater than 55° C., or greater than 60° C.

In some embodiments, the organism in the second bioreactor is capable of utilizing the volatile fatty acid produced by the first bioreactor as a carbon source. In some embodiments, the organism in the second bioreactor is capable of utilizing the volatile fatty acid produced by the first bioreactor to synthesize long chain fatty acids. In some embodiments, the organism in the second bioreactor is capable of utilizing the volatile fatty acid produced by the first bioreactor to synthesize triacylglycerides (also known as triglycerides or TGAs). In some embodiments, the organism in the second bioreactor is an anaerobe. In some embodiments, the organism in the second bioreactor is a eukaryote. In some embodiments, the organism in the second bioreactor is a yeast. In some embodiments, the second organism is Yarrowia lipolytica.

In some embodiments of the present invention, the second organism is genetically modified to enhance lipid production. In some embodiments, the lipids synthesized by the second organism comprise triglycerides (also known as triacylglycerides or TGAs). In some embodiments, the second organism comprises genetic modifications comprising upregulation of one or more genes whose products are responsible for pushing carbon flux into the pathway leading to lipid synthesis. In some embodiments, the second organism comprises genetic modifications comprising upregulation of one or more genes whose products are responsible for pulling carbon flux through the pathway leading to lipid synthesis. In some embodiments, the second organism comprises genetic modifications comprising upregulation of one or more genes whose products are responsible for pushing carbon flux into the pathway leading to lipid synthesis, and upregulation of one or more genes whose products are responsible for pulling carbon flux through the pathway leading to lipid synthesis. In some embodiments, the second organism comprises genetic modifications comprising increased expression of acetyl-coenzyme A carboxylase and/or increased expression of diacylglycerol acyltransferase, thereby increasing the efficiency of synthesis of triacylglycerides. In some embodiments, the methods disclosed achieve the synthesis of triacylglycerides by the second organism where the triacylglycerides comprise fatty acids groups that are less than 50% saturated fatty acid groups, or less than 45% saturated fatty acid groups, or less than 40% saturated fatty acid groups. or less than 35% saturated fatty acid groups or less than 30% saturated fatty acid groups or less than 25% saturated fatty acid groups or less than 20% saturated fatty acid groups or less than 15% saturated fatty acid groups, or less than 10% saturated fatty acid groups. In some embodiments, the methods disclosed provides a mixture of triacylglycerides comprising fatty acids groups wherein oleate (C18.1), linolinate (C18.2) and palmitoleate (C16.1) amount to more than 50% of the fatty acid groups comprising the TGAs. In some embodiments, the methods disclosed provides a mixture of triacylglycerides comprising unsaturated fatty acids groups that comprise more than 60% of the fatty acids comprising the TGAs synthesized by the second organism.

In some embodiments, the methods of culturing the first organism comprise an initial growth phase, characterized by cell mass expansion. In some embodiments, an initial growth phase is followed by a transition phase characterized by a change in the source and/or mixture of reducing agents in the feed stream, and/or by changes or modifications to the medium, and/or by steps undertaken to disinhibit production. Non-limiting examples include increasing the activity of enzymes promoting production of the volatile fatty acid, and/or reducing or removing inhibitors of production of the volatile fatty acid. In some embodiments, the initial growth phase and/or the transition phase are followed by a production phase comprising stable, high-density cell mass coupled with increased productivity of the volatile fatty acid.

In some embodiments, the initial expansion phase is fed with a feed gas comprising carbon monoxide as the reducing agent (source of electrons). In some embodiments, the production phase is fed with a feed gas comprising hydrogen as the reducing agent (source of electrons). In some embodiments, the production of volatile fatty acid in the production phase is controlled to achieve between 20 and 30 grams per liter of culture volume in the first bioreactor. In some embodiments, the productivity of the volatile fatty acid in the first bioreactor achieves greater than 0.8 grams per liter per hour. In some embodiments, the transition phase between cell expansion phase and production phase comprises a partial replacement of the bioreactor culture medium with fresh medium.

In some embodiments, the transition phase comprises removing carbon monoxide from the bioreactor medium in order to disinhibit the activity of hydrogenase. In some embodiments, the transition phase comprises steps to increase and enhance the activity of hydrogenase. In some embodiments, where hydrogenase activity is improved with the switch to hydrogen as an electron donor in the feed gases, cell culture viability is maintained at, at least 80% or at least 85%, or at least 90%, or at least 95% or at least 100% of the level achieved in the cell expansion phase. In some embodiments, hydrogenase activity is controlled and increased by one or more genetic modifications, thereby increasing the energy flux from hydrogen to acetyl-CoA.

In some embodiments, the methods presently disclosed provide a means whereby the second bioreactor achieves a lipid titer of at least 15 grams per liter of bioreactor volume, or at least 20 grams per liter of bioreactor volume, or at least 25 grams per liter of bioreactor volume, or at least 30 grams per liter of bioreactor volume, or at least 35 grams per liter of bioreactor volume, or at least 50 grams per liter of bioreactor volume, or at least 75 grams per liter of bioreactor volume. In some embodiments, the methods disclosed enable production of lipid in the second bioreactor that reaches or exceeds 30% of the biomass, or that reaches or exceeds 40% of the biomass, or that reaches or exceeds 50% of the biomass, or that reaches or exceeds 60% of the biomass.

In some embodiments of the methods disclosed, carbon dioxide produced by the processes in the second bioreactor is used as part of the feed gas mixture for the first bioreactor. In some embodiments, the non-lipid biomass generated by the process in the second bioreactor is used as a cell culture media component for use in either or both of the first and/or second bioreactors.

In some embodiments, the nitrogen content of effluent of the first bioreactor process is controlled, thereby enhancing nitrogen-inhibited lipid production in the second bioreactor. In some embodiments, the energetic efficiency of acetate to lipid conversion in the integrated process is greater than 50% of the theoretical maximum energy conversion, or greater than 55% of the theoretical maximum energy conversion, or greater than 60% of the theoretical maximum energy conversion, or greater than 65% of the theoretical maximum energy conversion, or greater than 70% of the theoretical maximum energy conversion, or greater than 75% of the theoretical maximum energy conversion, when measured as the conversion of energy in the feed to lipid produced, where the lipid produced is modeled as tripalmitin. In some embodiments, the method disclosed facilitates the production of biodiesel from carbon dioxide in a manner providing net carbon capture by overall fixation of carbon dioxide as fuel oil.

### BRIEF DESCRIPTION OF THE DRAWINGS

FIG. 1 shows the disclosed integrated bioprocess: Schematic of two-stage lipid production process using microbial conversion. In stage 1, CO₂ is fermented to volatile fatty acid; in stage 2, this acid is converted in to lipids by an engineered strain.

FIGS. 2A and 2B show cell growth (FIG. 2A) and acetic acid production (FIG. 2B) using either CO or H₂ as reducing gas at a composition of 7/3 CO/CO₂ or 7/3 H₂/CO₂ and a flow rate of 1,000 sccm. The maximum cell density under H₂ (max OD of ˜2.5) is less than (¼) of that achieved with CO (max OD of ˜11) as electron donor. Substantial amounts of acetic acid (˜30 g/L) are produced under both conditions. Standard deviations from triplicate runs are presented.

FIGS. 3A-3C show time courses of (a) acetic acid concentration (FIG. 3A), (b) specific hydrogenase activity (FIG. 3B), and, (c) optical density (FIG. 3C) for two different methods of switching the gas composition in the anaerobic bubble column. Black symbols are for the case in which gas switch from CO/CO₂ (4/1) and flow rate of 1000 sccm to H₂/CO₂ (2/1) and same flow rate was accompanied with replacement of half of the reactor medium with fresh media. In the run represented by the white symbols a similar gas switch was carried out but without medium replacement. Standard deviations from triplicate runs are presented.

FIG. 4 shows fermentation characteristics of Y. lipolytica in semi-continuous mode during acetic acid consumption (high strength). Y. lipolytica produced lipids at a titer of 51 g/L with a productivity of 0.26 g/L/h and lipid content of 61%. Standard deviations from triplicate runs are presented.

FIG. 5 shows acetic acid (3% v/v) consumption (low strength) and lipid production by using Y. lipolytica in semi-continuous mode. The time courses show a lipid titer of 46 g/L with an overall productivity of 0.27 g/L/h and lipid content of 59%. Standard deviations from triplicate runs are presented.

FIG. 6 shows fatty acid distribution during bioreactor fermentation by using Y. lipolytica. C16 palmitate; C16.1 palmitoleate; C18 stearate; C18.1 oleate; C18.2 linoleate. Samples performed in triplicates.

FIG. 7 shows carbon balance of acetate consumption and lipid production. Input carbon as acetate and output carbon (lipid, non-lipid, by-products, and CO₂). The cumulative carbon mass balance is closed to within 5%, with CO₂ accounting for 54% of the total carbon products.

FIG. 8 shows theoretical predictions and experimental values of acetate consumed and CO₂ produced. A stoichiometric model was constructed to assess the efficiency of acetate utilization during the fermentation process, taking into account acetate consumed and CO₂ produced for both non-lipid biomass production (growth) and for lipid production, including the associated ATP and NADPH costs of these processes. The experimental CO₂ production and acetate consumption are within 10% of the expected theoretical values. Model detail is provided in Example 6.

FIGS. 9A-9B show cell growth of M. thermoacetica (FIG. 9A) and acetic acid production (FIG. 9B) of the integrated fermentation experiment. In all three phases gaseous substrate is provided in continuous mode, while liquid as batch mode in the first two phases. In the third phase both gas and liquid are in continuous mode. Successful gaseous substrate switch allowed acetic acid production (maximum concentration of ˜25 g/L) in the anaerobic reactor. This is achieved by continuously removing acetic acid while recycling M. thermoacetica cells through a hollow fiber membrane. Standard deviations from triplicate runs are presented.

FIG. 10 shows lipid titer and biomass production in integrated process (phase III). The time course of cell growth and lipid production (18 g/L titer and 36% lipid content) in the aerobic bioreactor. Standard deviations from triplicate runs are presented.

### DETAILED DESCRIPTION

In the quest for inexpensive feedstocks for the cost-effective production of liquid fuels, gaseous substrates were examined that could be made available at low cost and sufficiently large scale for industrial fuel production. Described herein is a new bioconversion scheme that effectively converts syngas, generated from gasification of coal, natural gas or biomass, into lipids that can be used for biodiesel production. An integrated conversion method comprising a two-stage system is presented. In the first stage, an anaerobic bioreactor converts mixtures of gases of CO₂ and CO or H₂ to acetic acid using the anaerobic acetogen Moorella thermoacetica. The acetic acid product is fed as substrate to a second bioreactor, where it is converted aerobically into lipids by an engineered oleaginous yeast, Yarrowia lipolytica. The process carried out in each reactor is first described and then an integrated system that produces microbial oil using synthesis gas as input is presented. The integrated continuous bench-scale reactor system produced 18 g/L of C16-C18 triacylglycerides directly from synthesis gas, with an overall productivity of 0.19 g/L/h and a lipid content of 36%. While suboptimal relative to the performance of the individual reactor components, the presented integrated system demonstrates the feasibility of substantial net fixation of carbon dioxide and conversion of gaseous feedstocks to lipids for biodiesel production. The system can be further optimized to approach the performance of its individual units so that it can be used for the economical conversion of waste gases from steel mills to valuable liquid fuels for transportation.

There are several new concepts deployed in this study that allowed the successful implementation of the integrated gas-to-lipids conversion process. First, the acetogen M. thermoacetica was employed as a model organism because of its very high autotrophic flux to acetyl-CoA which produces naturally acetate at very high rates and almost theoretical yields via the canonical Wood-Ljungdahl pathway. Additionally, the high temperature of operation (Tₒₚₜ 60° C.) of this thermophilic organism makes it of industrial interest as it would require less cooling of syngas prior to feeding the bioreactor (12) compared to the other model acetogens, Clostridium ljungdahlii (Tₒₚₜ 37° C.) (13), and Acetobacterium woodii (Tₒₚₜ 30° C.) (14). Prior work with M. thermoacetica (7) provided the basis of acetic acid production at high rates relevant to this study.

Second, a novel gas composition switch strategy is provided as an important part of the process required for achieving higher overall acetate productivity. Additionally, this result points out the importance of the physiological state of M. thermoacetica at the time of switching the gas composition. Optimization can identify suitable medium replacement, culture viability, or alternately modifying medium composition to achieve maximum titers, yields and productivities.

Third, as summarized in Table 2, the figures of merit obtained for the integrated system are lower than those achieved for the individual bioreactor units. Additionally, the overall energetic efficiency (from hydrogen to lipid and yeast) of the integrated system is 34.4%, compared to a maximum theoretical of 43% (considering yeast energy content) and 60.5% (assuming only hydrogen and tripalmitin are the energy carriers). Model detail on energy efficiency calculation is provided in Example 6. Therefore the process can be further optimized, such as by nitrogen control in the media of the two units.

Fourth, there is significant potential for reduction of CO₂ emissions in the integrated system compared to the single-stage systems. For example, the process in the first reactor is theoretically expected to consume 100.2 mole of CO₂ while aerobic lipid synthesis is theoretically expected to produce 49.22 mole of CO₂ (a cumulative balance for the whole operation). As such, the process actually fixes CO₂ using CO and/or H₂ as reducing agents. Carbon dioxide generated in the lipid producing aerobic reactor can be recycled and utilized by the anaerobic bacteria of the first stage, yielding a significant overall rate of CO₂ fixation. In the same vein, the non-lipid biomass could be used to supply the yeast extract used in the media of both reactors following extraction of the lipids.

The main limitation in biodiesel (the primary renewable alternative to diesel) production is feedstock availability and cost (15). It is unlikely that food-competing feedstocks will contribute significantly to the production of renewable transportation fuels (16). In the quest for a cheaper feedstock, synthesis gas offers attractive features. Also, steel mill gas and natural gas can be used as a cost effective feedstock (12, 17, 18, 19). As such, a syngas-to-liquids process represents an infrastructure-compatible platform that will be important for reducing dependence on fossil fuels. The gas to liquid concept described herein can provide biodiesel production from CO₂ in a way that does not impose demands on or changes of land use and is not confined by the need of access to carbohydrate feedstocks.

The present disclosure describes systems and methods by which feed gases comprising carbon dioxide, carbon monoxide, and hydrogen, can be utilized as bioreactor inputs to produce an organic molecule that can then be further anabolized in a bioreactor to produce lipids. Such lipids can then provide feedstock for conversion to biodiesel using processes known in the art.

In some embodiments, the feed gases comprise natural gas. In some embodiments, the feed gases comprise carbon dioxide and an electron donor. In some embodiments, the feed gases comprise synthesis gas or syngas, a mixture of carbon monoxide and hydrogen with variable carbon dioxide content produced as a byproduct or intermediate in numerous industrial processes. In one non-limiting embodiment, the syngas utilized is produced as a waste product of steel mills.

In some embodiments, the gases are first synthesized into an organic molecule that is a volatile fatty acid comprising methanoic acid (also known as formic acid), or acetic acid (also known as acetate or ethanoic acid), or propionic acid (also known as propanoic acid or propanoate), or butyric acid (also known as butanoic acid), or isobutyric acid (also known as 2-Methylpropanoic acid), or valeric acid (also known as pentanoic acid), or isovaleric acid (also known as 3-Methylbutanoic acid). In one embodiment, the volatile fatty acid is acetate synthesized by an acetogenic microorganism. In some embodiments, the microorganism of the first bioreactor is a thermophilic microorganism having an optimum growth temperature (Tₒₚₜ) of at least 40° Celsius, or of at least 45° C., or of at least 50° C., or of at least 55° C., or of at least 60° C., or of at least 65° C., such that hot feed gas streams can be utilized with less cooling than if less thermophilic organisms were used in the first bioreactor. In some embodiments, the organism used to synthesize the organic molecule in the first bioreactor is selected based on demonstration of a high autotrophic flux from the carbon source to the organic molecule of interest as the feedstuff for a second bioreactor. In one embodiment, the acetogenic organism used in the first bioreactors is Moorella thermoacetica.

In some embodiments, the initially synthesized organic molecule is further synthesized into lipids in an aerobic process step. In some embodiments, the aerobic process step is accomplished by the oleaginous yeast Yarrowia lipolytica.

In some embodiments, the two process steps are linked as a pair of bioreactors, with the product of the first bioreactor feeding the second bioreactor. In some embodiments, the cells of the organism in the first bioreactor are passed through a device permitting the separation of the cells from the culture supernatant, thus allowing the recycling of the cells into the first bioreactor and the passage of the first bioreactor's product to the second bioreactor. In some embodiments, the level of the volatile fatty acid in the first bioreactor is optimized to provide a value that is close to the maximum concentration of the volatile fatty acid in the effluent of the first reactor, while still avoiding substantive toxicity to the organisms in the first bioreactor by virtue of the concentration of the volatile fatty acid. In some embodiments, the cells of the organism in the second bioreactor are passed through a device permitting the separation of the cells from the culture supernatant. One skilled in the art will appreciate that doing so allows the recycling of the cells into the second bioreactor, and the removal of the product of the second bioreactor.

In some embodiments, the two bioreactor arrangement can be run continuously to provide an integrated gas-to-oil process scheme. In some embodiments, the expansion of the organisms in the first bioreactor is accomplished by feeding the first bioreactor with a carbon dioxide/carbon monoxide mixture. In some embodiments, the productivity of the organism in the first bioreactor is improved by feeding the first bioreactor with a carbon dioxide/hydrogen mixture. In some embodiments, the organisms in the first bioreactor demonstrate a change in growth phase from expansion of cell density under feeding with a carbon dioxide/carbon monoxide mixture to a production phase where cell density plateaus, but specific productivity is increased under feeding with a carbon dioxide/hydrogen mixture.

In some embodiments, the shift in cell culture phase from cell expansion to production in the first bioreactor is accompanied by a replacement of some or all of the cell culture medium in the first bioreactor. This promotes endogenous hydrogenase activity by removing carbon monoxide inhibition of hydrogenase, thereby maintaining the viability of the culture in the first bioreactor during the switch in electron donor in the feed gas from carbon monoxide to hydrogen. In some embodiments, hydrogenase activity is enhanced during the switch to hydrogen as an electron donor by gradually reducing the carbon monoxide level and increasing the availability of hydrogen over a period of time. In some embodiments, hydrogenase activity can be enhanced by alerting the expression of genes in the organisms in the first bioreactor to augment the constitutive production of hydrogenase or enhance the activity of hydrogenase. In some embodiments, hydrogenase activity can be enhanced by removing inhibitors of hydrogenase, including but not limited to the removal of carbon monoxide. In some embodiments, hydrogenase activity is increased approximately 10-fold, or 20-fold, or 30-fold, or 40-fold, as the culture is transitioned to hydrogen as an electron donor.

In some embodiments, the production by the first bioreactor of the organic molecule of interest (for example, but not limited to, acetate) after the culture is transitioned to hydrogen as an electron donor is greater than 0.6 grams per liter per hour, or greater than 0.8 grams per liter per hour, greater than 1.0 grams per liter per hour, greater than 1.2 grams per liter per hour, or greater than 1.5 grams per liter per hour.

In some embodiments of the methods disclosed, the first bioreactor is transitioned to hydrogen as the electron donor in batch mode prior to extraction of any effluent from the first bioreactor. This allows the concentration of the organic molecule of interest to rise to a concentration useful for the initiation of the process in the second bioreactor. In some embodiments, flow of effluent from the first bioreactor is allowed to feed the second bioreactor for a period in which no product is removed from the second bioreactor. This allows the level of lipid in the oleaginous microorganism to rise to a level where harvesting effluent provides efficient recovery of lipid. In some embodiments, the feed gas flow, cell recycling, and effluent harvest (feed for the second bioreactor) of the first bioreactor and the cell recycling and effluent harvest of the second bioreactor can be controlled to provide a stable continuous process in which gas is transformed to lipid by carbon flux through the double bioreactor train. In some embodiments, the carbon to nitrogen ratio of the feed stream to the second bioreactor is increased to provided improved yield as measured by grams of lipid per gram of total biomass produced in the second bioreactor, where total biomass is equal to the sum of lipid biomass and non-lipid biomass.

The presently disclosed invention includes methods capable of converting energy from hydrogen to yeast biomass that are at least 80%, or at least 85% , or at least 90% or at least 95% of the theoretical maximum energy conversion efficiency of hydrogen to yeast. In some embodiments, the invention disclosed includes methods capable of converting energy from hydrogen to lipid biomass that are at least 50%, or at least 60%, or at least 70% or at least 80%, or at least 90% of the theoretical maximum energy conversion efficiency of hydrogen to lipid (wherein the energy content of lipid is assumed to be manifested as pure tripalmitin).

In some embodiments of the present invention, the biomass byproduct of the processes, for example, the non-lipid biomass of the yeast produced in the second bioreactor, can be used to provide inputs for the cell culture media required for the first and second bioreactors. One skilled in the art would understand that this enhances the efficiency of the overall processes and thereby improves the economic yield of the processes disclosed.

In some embodiments of the present invention, carbon dioxide produced by the aerobic processes in the second bioreactor is incorporated into the feed gas for the first bioreactor. This provides enhanced carbon-capture, thereby improving the process to better than carbon-neutral, and thus provides enhanced economic utility of the process where reduction in carbon emissions as carbon dioxide and or carbon capture are desirable for economic and/or regulatory reasons, or are seen as desirable from the perspective of environmentally ethical development and production practices.

By combining syngas fermentation with lipid production, the disclosed methods demonstrate that syngas can be effectively converted into lipids, and that this integrated bioprocess has potential to be an economically viable technology for the production of alternative fuels. In addition, microbial conversion of gaseous compounds facilitates the mitigation of gaseous pollution and production of biofuels and commodity chemicals. The developed bioprocess is useful for practical application to new or retrofit syngas bioconversion. The proper integration of microbial process with engineering tools and materials improves the understanding of microbial conversion processes, and those skilled in the art will appreciate the many practical applications of the disclosed invention.

### EXAMPLES

### Materials & Methods

### Bacteria, Media, and Cultivation Conditions

The anaerobic acetogenic bacterium, M. thermoacetica (ATCC 49707) was grown at 60° C. following strict anoxic techniques in an enhanced culture medium containing (per liter) 1.4 g KH2PO4, 1.1 g K2HPO4, 2.0 g (NH4)2SO4, 0.5 g MgSO4·7H2O, 10 g yeast extract, 10 g morpholinoethanesulfonic acid (Mes), 20 mL ATCC 1754 PETC trace elements solution (www.atcc.org), and 10 mL 0.3% cysteine solution. In addition, 0.5 mL 0.2% resazurin was added to indicate the presence of oxygen.

The engineered Y. lipolytica ACC-DGA strain (2) is a po1g (genotype MatA, leu2-270, ura3-302:URA3, xpr2-332, axp-2; source, Yeastern Biotech), transformed strain with plasmid pMT065 expressing genes ACC and DGA encoding acetyl-CoA carboxylase and diacylglycerol acyltransferase, respectively. ACC1 and DGA1 are important genes responsible for the first and last steps of lipid synthesis. The coupling of ACC1 and DGA1 allows effective flux diversion toward lipid synthesis and creation of a driving force (the simultaneous push and pull of carbon flux toward TAG) by sequestering product formation in lipid bodies. This strain contains the LEU2 marker and an additional copy of both the ACC1 and DGA1 genes. ACC1 is under control of the hp4d promoter (minimal LEU2 promoter preceded by 4 copies of the UAS1B upstream activation sequence enhancer) and the DGA1 gene is under the control of the Yarrowia TEF [transcription elongation factor 1(alpha)] promoter with its associated intron. The engineered Y. lipolytica ACC-DGA strain was grown with the following media. Selective YNB-Leu agar plates contained (per liter) 1.7 g yeast nitrogen base (without amino acids), 0.69 g CSM (Leu-), 20 g glucose, and 15 g Bacto agar. YPD medium was prepared (per liter) with 20 g Bacto peptone, 10 g yeast extract, and 20 g glucose (Sigma-Aldrich). YPA medium was similar to YPD but contained 28 g/L sodium acetate (or 20 g/L acetate) instead of glucose. Bioreactor inoculum was first grown in an overnight tube culture with YPD medium to an OD of 3-5, followed by streaking onto a YNB-Leu Petri dish. Individual colonies were subcultured into a culture tube [3 mL YPA in a 15-mL culture tube, 200 rpm, 28° C., 24 h (Thermo Scientific MaxQ 4000 orbital shaker with an orbit of 1.9 cm)]. Once the OD reached 3-5, they were again subcultured into an Erlenmeyer flask at an initial OD of 0.1 [50 mL YPA in 250-mL flasks, 200 rpm, 28° C., 2 d (Thermo Scientific MaxQ 4000 orbital shaker with an orbit of 1.9 cm)]. The bioreactor was inoculated with exponentially growing cells to a final concentration of 5% vol/vol. Yeast extract, Bacto agar, and Bacto peptone were purchased from BD. Sodium acetate was purchased from Macron Fine Chemicals. All other chemicals used were purchased from Sigma-Aldrich. The ACC-DGA strain accumulated up to 62% lipids from glucose as a substrate through de novo synthesis at an overall volumetric productivity of 0.143 g·L−1·h−1 (5). Subsequent optimized bioreactor cultivations improved performance to 55 g/L titer, 0.707 g·L−1·h−1 volumetric productivity, 67% lipid content, and 0.234 g/g yield (5).

### Anaerobic Bubble Column Bioreactor Operation

M. thermoacetica was grown in a 1 L glass bubble column bioreactor (inside diameter of 4.5 cm, a height of 80 cm; G. Finkenbeiner Inc.) at 60° C. Gas composition was controlled with a four-channel mass flow controller, controlling the flow rates of H₂, CO, CO₂, and N₂. Bioreactor temperature was maintained at 60° C. with a heating blanket and temperature controller. pH was controlled by addition of 5 N sodium hydroxide or hydrochloric acid, determined by an Etatron DLX pH/ORP pump control system and a submersible pH electrode. The AALBORG digital mass flow controllers, temperature, and pH were all purchased from Cole-Parmer. Prepared medium (excluding cysteine) was autoclaved autoclaved at 121° C. for 25 min, transferred to the bioreactor, and purged with oxygen-free N₂ gas overnight to remove all traces of dissolved oxygen. Afterward, pH was adjusted to 6.0 unless otherwise stated, and syngas flow was initiated and continuously bubbled through the bioreactor. Cysteine solution (10 mL, 3% wt/wt) was added to the medium to remove residual oxygen. The bubble column bioreactor was inoculated at 5% v/v with a culture grown in a serum bottle with the media listed above.

### Syngas Composition

Initial anaerobic reactor experiments were conducted with feed gas composition that was maintained constant throughout the fermentation process. The cells were grown on a syngas mixture supplied at a total gas flow rate of 1,000 sccm and with compositions of CO/CO₂ (4/1), and H₂/CO₂ (4/1). In subsequent experiments, where the gas composition was changed midfermentation, cells were grown initially on CO/CO₂ (4/1) at 1,000 sccm. After cells reached stationary phase and the acetate production leveled off, the syngas composition and flow rate were adjusted as described below. At the same time, half of the media in the bioreactor were replaced with fresh media, and flow through a hollow fiber membrane was initiated to recycle cells back to the bioreactor. The working volume of the bubble column was maintained constant at 1 L after the media replacement.

### Stirred Tank Aerobic Bioreactor Conditions

Y. lipolytica was grown in a 2 L stirred tank bioreactor (New Brunswick Scientific). The pH and dissolved oxygen (DO) levels were monitored using a pH probe and a DO probe (Mettler-Toledo Ingold Inc.). The initial medium was composed of 30 g/L sodium acetate (Macron Chemicals), 2.5 g/L yeast extract (Difco Laboratories), 4.2 g/L yeast nitrogen base (without amino acids and ammonium sulfate; Amresco), and 2.4 g/L ammonium sulfate (Macron Chemicals) (C/N molar ratio of 20). DO was controlled to 20% saturation, and pH was controlled to 7.3 by addition of acetic acid, and were maintained with cascade control. During the first 70 h of growth, the feed consisted of 3% acetic acid and 15 g/L ammonium sulfate to provide sufficient nitrogen for generation of non-lipid biomass. Thereafter the feed consisted solely 3% v/v acetic acid. Additional acetate required to maintain an acetate concentration of approximately 15 g/L in the bioreactor was provided as a sodium acetate solution (300 g/L) as needed. A hollow fiber module with a pore rating of 0.2 μm and surface area of 290 cm2 (MiniKros Sample Plus Filter Module; Spectrum Laboratories) was continuously used during the reactor operation to recycle cells and maintain constant volume by removing the excess volume pumped into the reactor through the acetic acid feeding (20). The acetic acid solutions were prepared with glacial acetic acid (Acros Chemicals). A theoretical maximum of carbon accumulation is determined as lipid and non-lipid production on acetate (21, 22).

### Integrated Bioprocess System

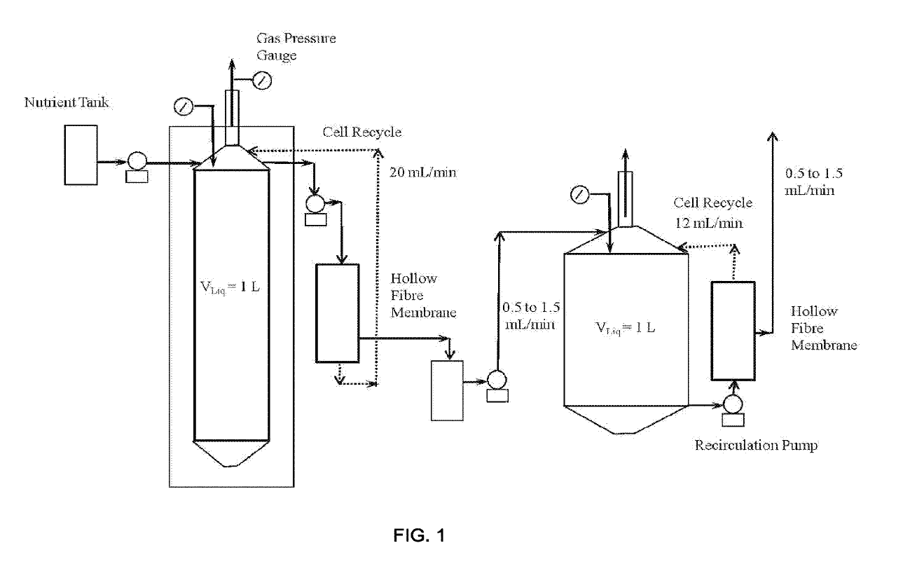

The bubble column bioreactor (for M. thermoacetica) and stirred tank bioreactor (for Y. lipolytica) were integrated in a process shown schematically in FIG. 1. Experimental conditions during integrated bioprocess run are provided in Table 1. In this system, gases are fed to the bubble column and are converted to acetic acid, which is, in turn, used as a feed to the stirred tank reactor and converted to lipids. The bubble column was operated first in a batch mode with respect to the liquid phase. A mixture of CO/CO₂ gases was supplied at a composition of 4:1 and a flow rate of 1,000 sccm. After 94 h, the gas composition was switched to H₂/CO₂ (2:1) and the flow rate increased to 1,200 sccm. At the same time, 500 mL of the medium was replaced by 500 mL fresh anaerobic media, using the hollow fiber membrane to retain cells in the bioreactor (MiniKros Sample Plus Filter Module; Spectrum Laboratories).

Following switch of syngas composition and attainment of stationary phase, continuous liquid removal was initiated and cells recycled via hollow fiber membrane. Once the culture reached stationary phase, at 185 h, flow of liquid media was initiated and the bubble column bioreactor with M. thermoacetica was switched to continuous operation with a flow rate of 0.5 mL/min. Cell recycle was also initiated at the same time from the hollow fiber membrane. Throughout the operation, pH was maintained at 6.0. Spent media from the hollow fiber membrane were transferred into the stirred tank bioreactor via a sterile container. The stirred tank bioreactor (for Y. lipolytica) was inoculated about 33 h after the switch of continuous operation, to allow enough time to feed ˜1,000 mL of acetate-containing media into the bioreactor. After 1 L effluent had been transferred, the aerobic bioreactor was inoculated and operated continuously, again with cell recycling accomplished via hollow fiber membrane. To keep the constant working volume in the bioreactor, the effluent containing low concentrations of acetic acid was removed via the hollow fiber membrane with a pore rating of 0.2 μm and surface area of 290 cm² (MiniKros Sample Plus Filter Module; Spectrum Laboratories). The liquid flow rates increased from 0.5 mL/min to 1.5 mL/min after the inoculation of Y. lipolytica.

### Fermentation Analytics

Effluent gas composition was analyzed via microGC-TCD. Acetate and citrate were measured via HPLC with RID detection. Growth was monitored via OD₆₀₀ for M. thermoacetica (23), and gravimetrically for Y. lipolytica. Conversion of OD to dry cell weight was via ratios determined in our laboratory. Total lipids were quantified using a modified version of a direct transesterification protocol adapted from U.S. Pat. No. 7,932,077 (24) and Griffiths et al. (25), using GC-FID. For details see supplementary notes (S4).

### Gas and Liquid Analysis

Gas flow rate was measured by bubble flow meter (Cole-Parmer). Gas composition was analyzed with a dual channel Agilent micro-GC equipped with PLOT U and Molecular Sieve columns and TCD detectors (Agilent Technology). Argon was used as the carrier gas. OD was measured at 600 nm with 1-mL cuvettes and an Ultrospec 2100 pro UV/Visible Spectrophotometer (General Electric). The OD of M. thermoacetica was proportional to DCW (correlation of ˜0.45 g dry cell per liter per unit OD660) (23), which value was confirmed. Y. lipolytica total biomass was determined gravimetrically, using cellulose nitrate (Whatman) filters. The filters were dried initially at 60° C. for 2 h and preweighed. A 15-mL volume of broth was poured into the holding reservoir fitted on the filter membrane. Vacuum was applied to pull the liquid through the membrane. The filters were dried for 24 h in a 60° C. oven and reweighed. Acetate and citrate were measured with a Waters Alliance 2695 HPLC with a Waters 410 Differential Refractometer and a Bio-Rad HPX-87H column (Waters Corporation). The column was eluted at 35° C. with 14 mM sulfuric acid as the mobile phase with a flow rate of 0.6 mL/min.

### Lipid Extraction and Quantification

Total lipids were quantified using a modified version of a direct transesterification protocol adapted from U.S. Pat. No. 7,932,077 (24) and Griffiths et al. (25). A correlation between the OD and cell biomass (0.3 g dry cell per liter per unit OD600) was used to determine the volume of cell culture. The volume of cell culture was chosen so that each sample would contain ˜1 mg of cell biomass. After centrifugation and removal of the supernatant, the cell pellet was suspended in 100 μL of hexane containing the internal standard glyceryl triheptadecanoate (C17 Tri-Acyl Glyceride). Subsequently, 500 μL of 0.5 N sodium methoxide was added to the mixture and the cell suspension vortexed at room temperature for 60 min. Next, 40 μL of 98% (wt/wt) sulfuric acid was added followed by addition of 500 μL hexane. This entire mixture was vortexed for 30 min at room temperature to extract the fatty acid methyl esters (FAMEs) into the hexane phase. Finally, the mixture was centrifuged at 8,000 rpm (Thermo Scientific MaxQ 4000 orbital shaker with an orbit of 1.9 cm) and the top hexane layer was transferred into vials for analysis through gas chromatography (GC). GC analysis of FAMEs was performed using a Bruker 450-GC instrument equipped with a flame-ionization detector and a capillary column HP-INNOWAX (30 m×0.25 mm) and the same oven conditions were used as discussed in Tai and Stephanopoulos (2). The FAME concentrations were calculated from commercial standards and normalized to the internal standard (methyl C17) in the control tube with no cells. Total lipid content was calculated as the sum of total fatty methyl ester contents for five FAMEs: methyl palmitate (C16:0), methyl palmitoleate (C16:1), methyl stearate (C18:0), methyl oleate (C18:1), and methyl linoleate (C18:2).

### Hydrogenase Assay

Hydrogenase activity was assayed anaerobically by monitoring the reduction of benzyl viologen with permeabilized cells in a sealed cuvette (26). An electron acceptor (EA) solution consisting of oxidized benzyl viologen and a cell solution (CS) sampled from fermentation were prepared in two separate Hungate tubes in an anaerobic chamber. The EA solution contained 0.3 mL of 1 M potassium phosphate buffer (pH 6), 0.4 mL of 40 mM benzyl viologen dichloride (BV), and 2.3 mL deionized water. The CS contained 0.3 mL of 1 M potassium phosphate buffer (pH 6), 0.3 mL of 0.5M DTT, 0.3 mL cell broth, and 1.8 mL deionized water. After preparation, both tubes were removed from the anaerobic chamber and purged with pure H2 gas at a flow rate of 50 sccm in a water bath at 60° C. for 5 min. One minute before the end of purging, 0.3 mL Triton X-100 solution was injected to the CS tube to permeate the cell wall and expose hydrogenase to the solution. To start the assay, 2 mL from the EA solution tube and 0.67 mL from the CS tube were mixed in a sealed anaerobic cuvette, and the reduction of BVox to reduced BVred was monitored continuously at 546 nm. The activity (U) was calculated from the initial slope, using an extinction coefficient of 7.55 m³·mol⁻·cm⁻¹. One unit was defined as the consumption of 1 μmol H₂ consumed per minute. Specific activity (U/mg) was determined by dividing the activity by the cell mass in the assay. It should be noted that the determined activity represents a global enzymatic activity of all hydrogenases involved in hydrogen uptake. Prior research has shown that several different hydrogenase enzymes are active in the acetogen M. thermoacetica (27).

### Example 1: Cell Growth and Acetate Production from Syngas

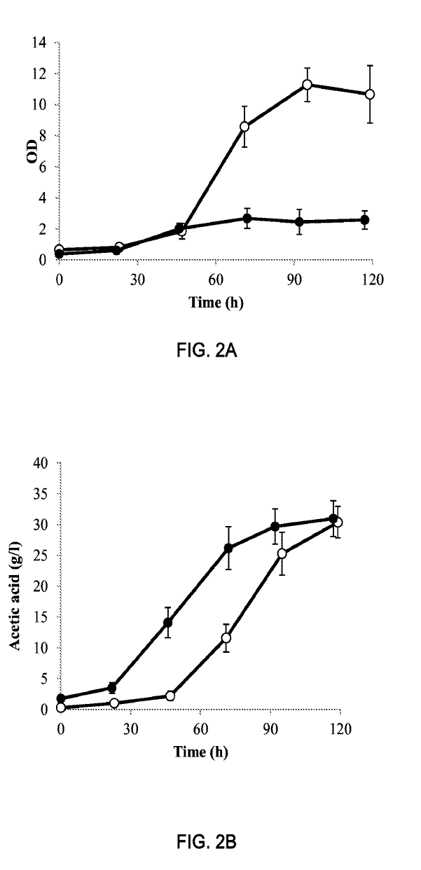

Acetic acid production from syngas by M. thermoacetica is shown in FIGS. 2A and 2B, which presents time courses for growth (FIG. 2A), and acetic acid titer (FIG. 2B) in an anaerobic bubble column run independently. Prior work provided the basis for flow rates relevant to this study (7). Fermentation was carried out at a flow rate of 1,000 sccm using either CO or H₂ as reducing gas at a composition of 7/3 CO/CO₂ or 7/3 H₂/CO₂. Substantial amounts of acetic acid (in excess of 30 g/L) were produced under both conditions during similar time periods. Notably, the maximum cell density under H₂ (max OD of ˜2.5) was less than ¼ of that achieved with CO (max OD of ˜11) as electron donor. Since the volumetric productivities of acetic acid were similar in these two experiments, the specific productivity of acetic acid under H₂/CO₂ was therefore 4 times greater than that under CO/CO₂. This difference in specific cell productivity is likely due to the energetic difference between H₂ and CO metabolism in this organism. M. thermoacetica generates ATP under autotrophic conditions by chemiosmosis. In this mechanism, oxidation of reduced ferredoxin by an energy-conserving hydrogenase complex (Ech) results in translocation of protons across the membrane. The generated proton gradient is used by the membrane-bound ATP synthase to generate ATP (8). Production of acetyl-CoA from H₂/CO₂ involves oxidation of 2 mols of ferredoxin for every mole of acetyl-CoA synthesized, whereas production of acetyl-CoA from CO requires oxidation of 6 mols of ferredoxin. Thus, growth on CO supports more ATP production, which, in turn, translates into higher biomass concentrations. The apparent ATP limitation during growth on H₂/CO₂ forces increased diversion of acetyl-CoA from biomass synthesis to acetate production to increase ATP synthesis via substrate level phosphorylation, resulting in higher overall acetate fluxes per unit biomass.

### Example 2: Acetate Productivity with Gas Composition Switch and Media Replacement

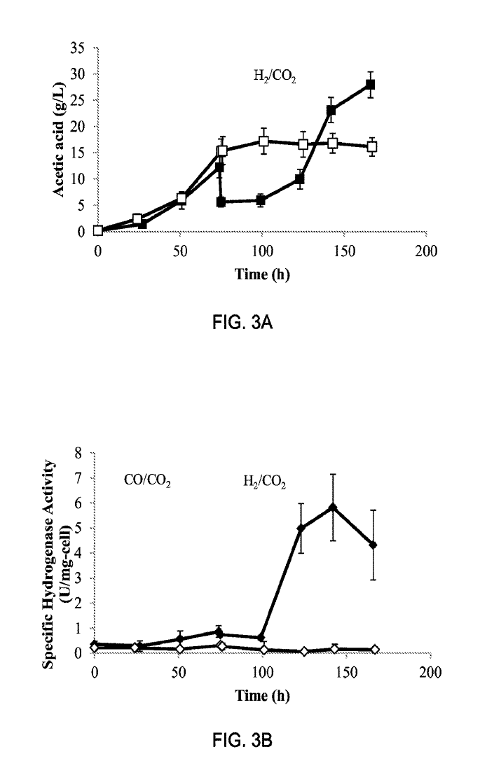

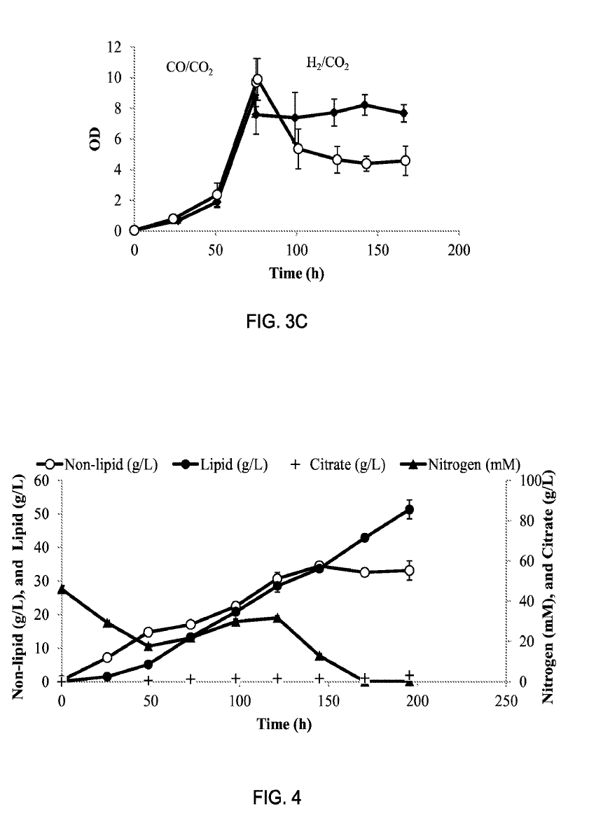

As shown in the previous section, the specific productivity of acetate on H₂ by M. thermoacetica is about 4 times that achieved on CO. On the other hand, much higher cell densities can be supported by a CO/CO₂ mixture. This suggests that one should first grow the M. thermoacetica culture on a CO/CO₂ mixture and switch to a H₂/CO₂ composition after the culture is established to take advantage of the higher acetate specific productivity on hydrogen. An experiment was thus designed to assess whether the high cell density achievable by growth on CO/CO₂ could be harnessed for higher overall productivity by switching the gas composition to H₂/CO₂ after the initial growth phase. However, the simple switch of gas composition led to a significant decline of M. thermoacetica culture and no change in acetic acid production (FIGS. 3A-3C). We hypothesized that this might be due to low hydrogenase activity at the time of the switch which is known to be inhibited by carbon monoxide (FIG. 3B). Though constitutively expressed, hydrogenase activity is 18-fold higher in H₂-cultivated cells than CO-grown cells (9), and it is required for growth on H₂ as a sole electron donor. Successful transition from CO to hydrogen without decline in cell mass and which also maintained the biosynthetic capacity of cells was achieved when half the medium was replaced with fresh medium at the same time of the gas switch. In this experiment, after an initial decline caused by the dilution from the fresh medium, cell density was stabilized (FIG. 3C) at the new value following the gas switch and, more importantly, maintained its acetic acid productivity. An average overall acetic acid productivity of 0.9 g/L-hr after the gas switch was obtained, which was 50% higher than that of 0.6 g/L-hr observed before the gas switch. These results are consistent with the time course of hydrogenase activity, which rose roughly 20-fold in one day following the replacement of CO by H₂. This experiment demonstrated that the volumetric productivity of acetic acid could be significantly improved by switching the gas composition and replacing the media mid-run, relatively to what was possible before using CO₂/H₂ or CO₂/CO gas alone.

### Example 3: Lipid Production from High Strength Acetic Acid by Y. lipolytica

Lipid production from acetic acid (30% v/v) by Y. lipolytica in a fed-batch mode is shown in FIG. 4. Y. lipolytica produced lipids at a titer of 51 g/L with a productivity of 0.26 g/L/h and lipid content of 61%, which is the highest reported to-date on acetate. Fontannile et al (2012) grew Y. lipolytica first on glucose and then transferred to volatile fatty acids (VFAs), obtaining lower productivity (0.16 g/L/h) compared to our study, albeit at lower lipid titers (12 g/L), conversion yields (0.13 g/g) and lipid content of 40% (10).

### Example 4: Lipid Production from Low Strength Acetic Acid by Y. lipolytica

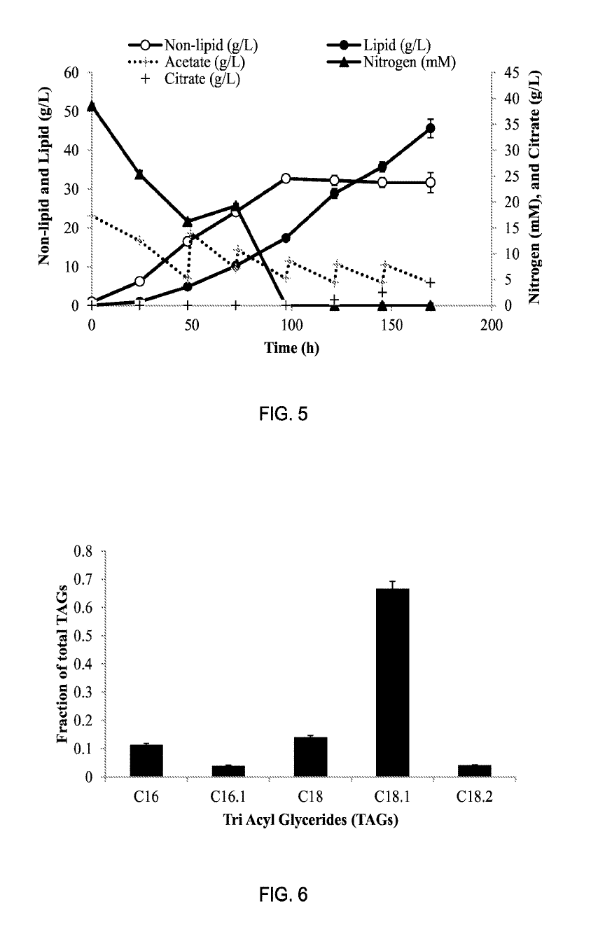

A dilute substrate will result in a dilute product. An exception is possible in the case of intracellular products (like lipids) and also when cells are recycled in order to increase their residence time in the bioreactor to allow them more time to attain higher lipid content. This can be achieved with the use of a hollow fiber membrane whereby spent medium with low concentration of residual acetate is removed whereas cells are recycled back in the bioreactor. This scheme was implemented and FIG. 5 shows the time courses of lipid production from dilute acetic acid (3% v/v). A lipid titer of 46 g/L was obtained with an overall productivity of 0.27 g/L/h and lipid content of 59%. An average lipid composition profile generated in this run is shown in FIG. 6. This profile is similar to that of Knothe (11), with higher fractions of unsaturated fats, which are desirable as they increase the cetane number and decrease the cloud point of the resulting biodiesel.

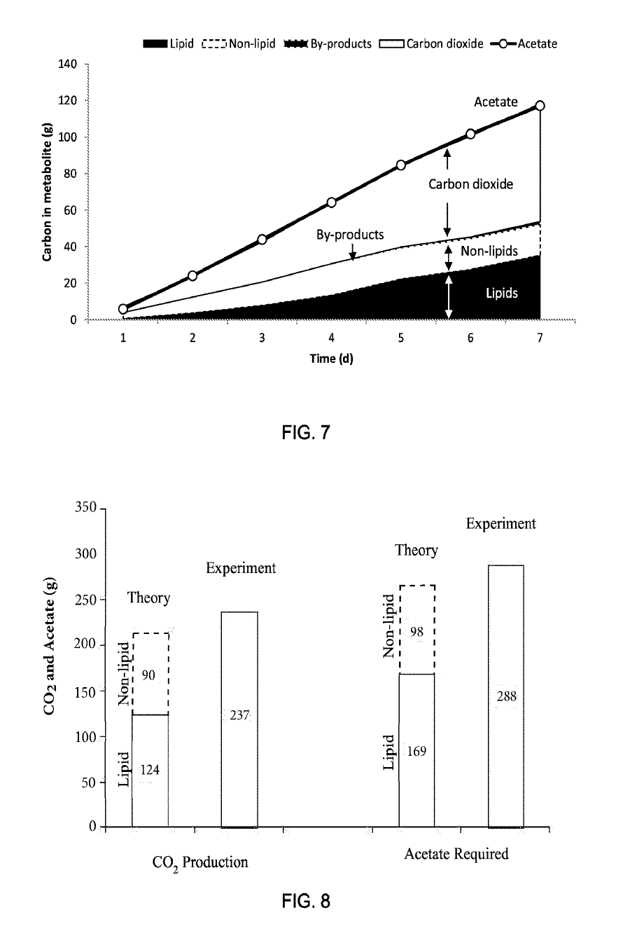

Carbon fluxes were calculated for this run. Y. lipolytica consumes acetic acid and converts the carbon to CO₂, lipids and non-lipid biomass, with small amounts of citrate produced as by-product. The cumulative carbon mass balance is closed to within 5%, with CO₂ accounting for 54% of the total carbon products (FIG. 7). A stoichiometric model was constructed to assess the efficiency of acetate utilization during the fermentation process considering usual metabolic processes and associated ATP and NADPH costs for non-lipid biomass production (growth) as well as lipid production. Details of the model are provided in Example 6. The model essentially predicts, for a given amount of lipid and biomass formed, the amounts of acetate required and CO₂ formed, based on reasonable assumptions about the pathways responsible for the main metabolic processes underlying growth and lipid synthesis. By substituting the experimentally measured lipid and non-lipid production data, we determined the theoretically predicted amount of acetate consumption and carbon dioxide production. Upon comparison with the respective experimentally observed levels of acetate consumption and CO₂ production it can be seen that experimental lipid and non-lipid production are within 10% of the theoretical values, as shown in FIG. 8. This suggests that the engineered strain is well optimized for lipid synthesis, wasting very little substrate for other processes.

### Example 5: Integrated System for Syngas Bioconversion to Lipids

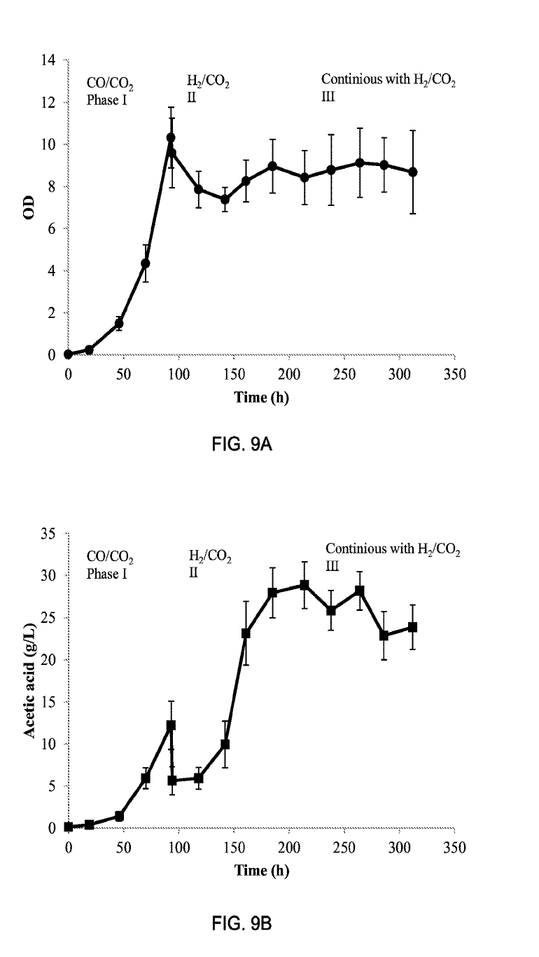

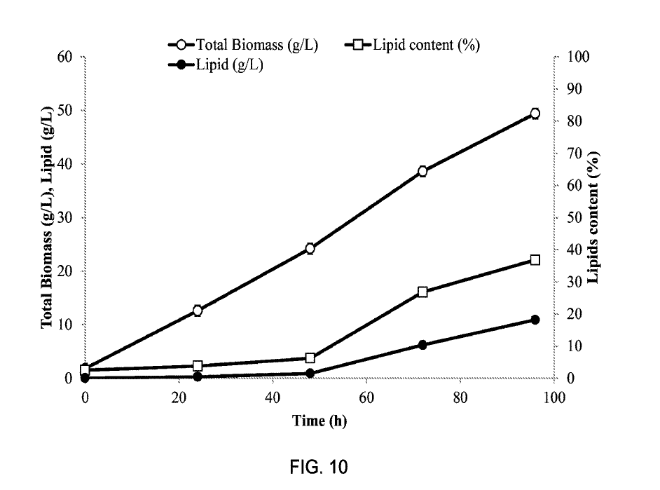

Based on the results obtained with the two bioreactor units, the integrated system of FIG. 1 was assembled and operated as shown in Table 1. FIGS. 9A and 9B shows the time courses for cell growth (FIG. 9A) and acetic acid production (FIG. 9B) by M. thermoacetica in the anaerobic bubble column, with FIG. 10 summarizing the same for Y. lipolytica in the aerobic stirred tank bioreactor. Before 93 hours, (first phase), the anaerobic bioreactor was fed with a CO/CO₂ gas mixture at 1,000 sccm and composition of (4/1). During this period, M. thermoacetica grew to OD of 10.3 and acetic acid accumulated to 12 g/L with a productivity of 0.4 g/L-hr, excluding the lag phase. Then, the gas supply was changed to H₂/CO₂ at a flow rate of 1,200 sccm and composition of (2/1). The second phase lasted from 93 hours to 218 hours and was characterized by a steady increase of acetic acid until the maximum of 25 g/L was reached. After the gas switch, the cell OD dropped from the maximum value of 10.3 to about 8 and recovered slowly until the end of the second phase (see supplementary notes S2 for different media replacement ratios). At the same time (186 to 218 hours), ˜1000 ml liquid medium was removed from the anaerobic bioreactor through a hollow fiber membrane and fed into the aerobic bioreactor. In the third phase (after 218 hours), continuous flow operation was initiated, which maintained the level of acetic acid in the anaerobic reactor at the maximum concentration of ˜25 g/L. This was achieved by continuously removing acetic acid while recycling M. thermoacetica cells through a hollow fiber membrane. Cell growth was minimal during this phase and the reactor thus operated like a perfusion bioreactor. The maximum acetic acid concentration of 25 g/L was selected to prevent inhibition of further acetate production (7). The working volumes of both bioreactors were maintained constant by the addition of fresh media in the first bioreactor and removal of spent media from the aerobic bioreactor as shown in FIG. 1. The acetic acid concentration of media reservoir was measured periodically between 118 and 312 hours, with a total of 212 g of acetic acid produced in 194 hours. The average productivity of acetic acid was 1.1 g/L-hr after the gas switch, 3 fold higher than the first stage when CO/CO₂ was used as gas feedstock.

The time courses of cell growth and lipids production in the aerobic bioreactor are presented in FIG. 10. Lipid titer was 18 g/L and lipid content 36%. Although these figures are lower than those achieved in the individual units and prior studies (2), the results are nevertheless encouraging as they demonstrate the feasibility of operating an integrated gas-to-lipids process, which was the goal of this study. A comparison of the results obtained from the single stage bioreactors and those of the combined process is provided in Table 2. The main difference between the independent lipid production run and the integrated process is the lipid yield from acetic acid. In the single unit investigation, more acetic acid was converted to lipids, while in the combined process more acetic acid was converted to non-lipid biomass. In Y. lipolytica, the C/N ratio is known to strongly influence lipid production and titers. In the combined process, the C/N ratio was not strictly controlled and this likely impacted the yields negatively. Therefore, improvements should aim to minimize the nitrogen content of the M. thermoacetica medium, which will limit the nitrogen content of the acetic acid feed to the Y. lipolytica reactor after the start of the lipid production phase.

### Example 6. Estimation of Theoretically Possible Energy Yield

A theoretical maximum of carbon accumulation in lipids is calculated based on microbial metabolism to determine stoichiometry of lipid and nonlipid production on acetate. The objective of this calculation is to determine the efficiency of the oleaginous microbe in converting acetate to lipids during the fermentation process. For the microbe to accumulate lipids, it has to first generate nonlipid biomass from acetate and then divert subsequent carbon toward lipid accumulation. Thus, acetate consumed by the microbes would be diverted toward one of two end products: nonlipid biomass and lipids. Whether the microbe is consuming more than the theoretically required amount of acetate (carbon feedstock) or producing more than the theoretically required amount of carbon dioxide (lost carbon), to produce the observed amount of lipids and nonlipids during the fermentation was investigated. To this end, a model was first developed that describes the stoichiometry of conversion of acetic acid to the respective product.

### Lipid Production on Acetic Acid

The stoichiometry of lipid production from acetic acid was obtained by accounting for (i) acetic acid required to produce tripalmitin (model lipid, a triglyceride form of palmitic acid), (ii) acetic acid required to produce NADPH (reducing equivalent) to sustain lipid production, and (iii) acetic acid required to produce ATP to sustain lipid production. The final equation relating acetic acid to lipid production is

50.1 CH₃COOH→49.2CO₂+C₅₁H₉₈O₆. [S1]

The equation above provides the theoretical amounts of acetic acid required and carbon dioxide generated during the production of 1 mol of tripalmitin. Details of its derivation are provided below under Stoichiometry for Acetate Conversion to Lipids. Using this equation and the molecular weights of acetate and tripalmitin, the maximum theoretical yield of lipid production from acetate was calculated at 0.272 g lipid per gram acetate.

### Nonlipid Biomass Production on Acetate

Similarly, a model for the production of nonlipid biomass from acetate was developed, which takes into account (i) the chemical formula of nonlipid biomass, (ii) the theoretical dry cell weight produced per electron, and (iii) the acetate consumed for ATP production. The final equation is

6.3 CH₃COOH→7.7CO₂+C₅H_{8.3} O_{2.7}N_{0.7}. [S2]

Thus, we obtain a stoichiometric equation that relates nonlipid biomass production to the moles of acetate consumed and moles of CO₂ produced. By plugging in the observed measurements of the lipid and nonlipid amounts into Eqs. S1 and S2, respectively, one can obtain the theoretical levels of acetic acid consumption and carbon dioxide production associated with each conversion. The theoretical estimates of acetic acid consumed and carbon dioxide produced are compared in to the observed experimental quantities to determine the extent of inefficiency in the microbial metabolism during the fermentation.

### Details of the Models

### Stoichiometry for Acetate Conversion to Lipids

Biosynthesis of triglycerides from acetate requires several pathways. Gluconeogenesis ultimately provides the glycerol backbone. As the starting substrate for gluconeogenesis is a four-carbon compound (oxaloacetate/pyruvate), anaplerotic pathways such as the glyoxalate cycle or the methyl-citrate cycle are required when acetate is the substrate. Fatty acid biosynthesis catalyzes the ATP and NADPH-dependent conversion of acetate to palmitic acid, and finally the Kennedy pathway condenses glycerol with the lipids to produce the triacylglycerides. All of the above pathways, as well as pathways for NADPH and ATP generation, are taken into consideration to arrive at the final equation for acetate to lipids. Note that the ATP-generating equation is also used in the prediction of the biomass stoichiometry in Stoichiometry for Acetate Conversion to Nonlipid Biomass below.

The reactions involved in this process have been grouped into modules or sets for simplicity. The symbols (c) and (m) refer to whether the metabolite exists in the cytosol or in the mitochondria. The following modules of reactions have been assumed: (i) acetate to acetyl-CoA (ACA), (ii) ACA to oxaloacetate (OAA), (iii) OAA to glycerol, (iv) ACA to C16 fatty acid, and (v) (fatty acid+glycerol) forms lipid.

The equations of each of the above modules were analyzed.

### Set (i)

Acetate+ATP→Acetyl-CoA(Acetyl-CoA Synthetase). [S3]

This reaction proceeds through the action of the enzyme Acetyl-CoA Synthetase (ACS) in the cytosol.

### Set (ii)

Gluconeogenesis occurs in the cytosol and hence oxaloacetate needs to be present on the cytosol side to initiate gluconeogenesis. This process occurs through successive reactions. First, the glyoxalate cycle operates and produces a succinate for every two acetyl-CoA molecules. Next, succinate produced in the cytosol permeates through the mitochondrial membrane and enters the TCA cycle operating in the mitochondria. Succinate converts to malate, which permeates out into the cytosol. Cytosolic malate is acted upon by the malic enzyme to produce pyruvate, which ultimately is acted upon by the enzyme pyruvate carboxylase to form oxaloacetate in the cytosol. The reactions are as follows:

2ACA (c)→succinate (c)+NADHc (glyoxalate cycle) (28)

Succinate (c)→succinate (m) (succinate exit)

Succinate (m)→malate (m)+FADH₂ (TCA cycle) (28)

Malate (m)→malate (c) (malate exit)

Malate (c)→pyruvate (c)+NADPHc+CO₂ (malic enzyme) (28)

Pyruvate (c)+CO₂+ATP→OAA (c) (pyruvate carboxylase) (28)

The net reaction amounts to:

2ACA (c)+ATP→OAA (c)+NADHc+FADH₂+NADPHc. [S4]

### Set (iii)

This accounts for the sum of reactions leading up to glycerol production from oxaloacetate via gluconeogenesis (28):

OAA (c)+GTP+ATP+NADHc→Glycerol+CO₂ (gluconeogenesis). [S5]

### Set (iv)

This accounts for the set of reactions leading up to palmitic acid production from acetyl-CoA via the fatty acid synthesis pathway (29):

24ACA (c)+18NADPH+24NADHc+69ATP→3 palmitic acid (fatty acid synthesis) [S6]

### Set (v)

This accounts for the tripalmitin production via the Kennedy pathway (28):

Glycerol (c)+3 palmitic acid+ATP→tripalmitin (Kennedy pathway). [S7]

Once the reaction modules have been analyzed to obtain a single reaction, the next step involves adding these to obtain our final equation. Adding sets (ii) through (v) (reactions 2-5) provides the following equations:

2ACA (c)+ATP→OAA (c)+NADHc+FADH₂+NADPHc [S4]

OAA (c)+GTP+ATP+NADHc→glycerol+CO₂ (gluconeogenesis) [S5]

24ACA (c)+18NADPH+24NADHc+69ATP→3 palmitic acid [S6]

Glycerol (c)+3 palmitic acid+ATP→tripalmitin (Kennedy pathway) [S7]

with the net equations being:

26ACA(c)+17NADPHc+72ATP+GTP+24NADHc→tripalmitin+CO₂+FADH₂. [S8]

At this point, the acetate required to provide the required NADPH, NADH, and ATP needs to be accounted for. It is assumed that all of the NADPH is provided through the action of the malic enzyme. The following equation accounts for NADPH production:

NADHc+ATP→NADPHc (malic enzyme). [S9]

ATP production from cytosolic ACA is not as straightforward as ACA cannot cross the mitochondrial membrane to form mitochondrial ACA, which could then directly enter the TCA cycle. Thus, the glyoxalate cycle operates to form succinate, which eventually forms pyruvate. Pyruvate can permeate through the mitochondria and then decarboxylate to form acetyl-CoA, which subsequently enters the TCA cycle. The equation for ATP/NADH production through the TCA cycle from cytosolic acetyl-CoA proceeds through the following set of equations:

2ACA (c)→succinate (c)+NADHc (glyoxalate cycle)

Succinate (c)→succinate (m) (succinate exit)

Succinate (m)→malate (m)+FADH₂ (TCA cycle)

Malate (m)→malate (c) (malate exit)

Malate (c)→pyruvate (c)+NADPHc+CO₂ (malic enzyme) (28)

Pyruvate (c)→pyruvate (m) (pyruvate exit)

Pyruvate (m)→3CO₂+4NADHm+FADH₂+GTP (TCA cycle) (28)

4NADHm→4NADHc (29)

The net equation is

2ACA (c)→4CO₂+5NADHc+2FADH₂+GTP+NADPHc. [S10]

Eq. S10 can also be used to produce more ATP through the conversion of the NADH and FADH₂ in the electron transport chain. However, it also is assumed that the NADPH would not be oxidized because there is a high demand for NADPH in the cell during the fatty acid synthesis as a reducing agent (1NADH=2.5ATP, 1FADH2=1.5ATP, 1GTP=1ATP). The equation becomes

2ACA (c)→4CO₂+16.5ATP+NADPHc. [S11]

Eqs. S8-S11 add up together to arrive at the final equation for lipid production from acetate. The strategy used is to assume Eqs. S9-S11 are multiplied by unknowns x, y, and z and added to Eq. S8. The equations are added such that the coefficients of NADH, NADPH, and ATP add up to 0. In this way, one can ensure that the equation accounts for the exact amount of acetate needed to arrive at 1 mol of tripalmitin:

26ACA (c)+17NADPHc+72ATP+1GTP+24NADHc→Tripalmitin+CO₂+FADH₂. [S8]

xNADHc+xATP→xNADPHc (Multiplying [S9] by the unknown x)

2yACA (c)→4yCO₂+5yNADHc+2yFADH₂+yGTP+yNADPHc. (Multiplying [S10] by the unknown y)

2zACA (c)→4zCO₂+16.5zATP+zNADPHc. (Multiplying [S11] by the unknown z)

these three equations then add up to:

(26+2y+2z)ACA+(17−x−y−z)NADPH+(71.5+x−4y−16.5z)ATP+(24+x−5y)NADH→(4y+4z+1)CO₂+tripalmitin (converting the FADH₂ and GTP into ATP). [S12]

However, the substrate is acetate and hence, multiplying Eq. S3 by (26+2y+2z) and adding to [S12] would substitute all of the ACA by acetate:

(26+2y+2z)acetate+(17−x−y−z)NADPH+(71.5+x−4y−16.5z+26+2y+2z)ATP+(24+x−5y)NADH→(4y+4z+1)CO2+tripalmitin. [S13]

Assigning the coefficients of NADPH, NADH, and ATP to be 0, a system of three equations in x, y, and z is obtained:

17−x−y−z=0 (coefficient of NADPH)

24+x−5y=0 (coefficient of NADH)

97.5+x−2y−14.5z=0 (coefficient of ATP).

Solving for the above three equations, x=4.944, y=5.788, and z=6.267 are obtained. Putting the above values of x, y, and z into Eq. S13, the final equation for conversion of acetate into lipids is obtained as:

50.11 acetate→49.22CO₂+tripalmitin (Eq. S1).

### Stoichiometry for Acetate Conversion to Nonlipid Biomass

Nonlipid biomass of average yeasts has a chemical formula of C₅H_{8.3}O_{2.7}N_{0.7}. It corresponds to a molecular weight of 121.3 g/mol. Shuler and Kargi (30) provided an estimate for the yield of dry cell weight (DCW) per electron available: 3.14 g DCW per electron. Because acetic acid has eight electrons, it would correspond to a biomass (nonlipid) yield of 3.14×8=25.1 g DCW per mole acetic acid. With the molecular weight of average yeast biomass known, the conversion can be shown as

CH₃COOH→0.21C₅H_{8.3}O_{2.7}N_{0.7}+0.95CO₂. [S14]

Here the moles of CO₂ were obtained by subtracting the carbon moles in biomass from the carbon moles in acetic acid (=2−0.21×5). In addition, there would be ATP expended by the cell for biomass production. According to Verduyn (31) 105.5 mmol ATP is required per gram dry biomass, which translates to the fact that for every 0.21 mol of biomass produced, 2.65 mol ATP would be required. Thus, Eq. S14 would be modified as:

CH₃COOH+2.65ATP→0.21C₅H_{8.3}O_{2.7}N_{0.7}+0.95CO₂. [S15]

Accounting for the additional acetic acid required for producing 2.65 mol of ATP (described in Stoichiometry for Acetate Conversion to Lipids) and normalizing the equation to 1 mol of biomass, the following is obtained:

6.3 CH₃COOH→7.7CO₂+C₅H_{8.3}O_{2.7}N_{0.7} (Eq. S2).

### Energy Efficiency Calculations

### Experimental Energy Efficiency Using the Lower Heating Value

Where Lower heating value (LHV) are taken as follows:

| Compound | LHV (MJ/kg) | Amount Produced (g) |
|---|---|---|
| Hydrogen | 120.21 | −28 |
| Tripalmitin | 36.48 | 18 |
| Yeast | 15.7 | 32 |

Energy efficiency=(18×36.8+15.7×32)=(28×120.21)=34.4%.

### Theoretical Maximum Energy Efficiency Treating only Lipid as the Product

Hydrogen LHV: −242.82 kJ/mol (cta.ornl.gov/bedb/appendix_a/Lower_and_Higher_Heating_Values_of_Gas_Liquid_and_Solid_Fuels.pdf).
Tripalmitin heat of combustion HHV: −31606 kJ/mol (webbook.nist. gov/cgi/cbook.cgi?ID=C555442&Mask=2)

The heat of combustion for tripalmitin is the higher heating value (HHV). To convert heat of combustion to LHV, account for water's heat of vaporization:

Liquid water: Δ_{f}H°liquid=−285.83 kJ/mol

Gaseous water: Δ_{f}H°gas=−241.83 kJ/mol

Water: Δ_{f}H°=−241.83−(−285.83)=44 kJ/mol; H₂O (liquid); H₂O (g) (this 44 is used below).

HHV of tripalmitin: Δ_{c}H°liquid:

=−31,606 kJ/mol C₅₁H₉₈O₆;C₅₁H₉₈O₆+72.5O₂→51CO₂+49H₂O (1).

LHV of tripalmitin: Δ_{c}H°liquid:

=−31,606+(44)×(49H₂O)

=−29,450 kJ/mol C₅₁H₉₈O₆; C₅₁H₉₈O₆+72.5O₂→51CO₂+49H₂O (g).

This gives:

Hydrogen LHV: −242.82 kJ/mol

Tripalmitin LHV: −29,450 kJ/mol.

The absolute maximum (no biomass production):

$$\frac{\begin{matrix}50.1 \times [ 4 {H}_{2} + 2 {CO}_{2} = > acetate ] \\ 50.1 acetate = > tripalmitin\end{matrix}}{50.1 \times 4 hydrogen = > tripalmitin}$$

Absolute max (stoichiometric) energy efficiency:

(−29,450×1 tripalmitin)/(−242.82×50.1×4 hydrogen)=60.5%.

### Theoretical Maximum Energy Efficiency Treating Lipid and Yeast Biomass as the Product

Using the experimental ratio of produced lipids to biomass,

$$\frac{\begin{matrix}50.11 acetate = > 49.22 {CO}_{2} + tripalmitin \\ 50.11 \times [ 4 {H}_{2} + 2 {CO}_{2} = > acetate ]\end{matrix}}{50.11 \times 4 hydrogen = > tripalmitin ( 807.34 kg \text{/} kmol ) \\}$$

$$\frac{\begin{matrix}6.3 acetate = > 7.7 {CO}_{2} + {C}_{5} {H}_{8.3} {O}_{2.7} {N}_{0.7} \\ 6.3 \times [ 4 {H}_{2} + 2 {CO}_{2} = > acetate ]\end{matrix}}{6.3 \times 4 hydrogen = > {C}_{6} {H}_{8.3} {O}_{2.7} {N}_{0.7} ( 121.42 kg / kmol )}$$

### Experimental:

Biomass=32 g=264 mmol

Lipids=18 g=22.3 mmol

So the experimental equation is (where the hydrogen is theoretically calculated from the theoretical equations above and the amount of biomass and lipids produced):

11,123 hydrogen=>264C₅H_{8.3}O_{2.7}N_{0.7}+22.3 tripalmitin.

Hence theoretical energy efficiency (LHV) is

[(264 mol biomass)(−1,909.2524 kJ/mol)+(22.3 mol tripalmitin)(−29,450 kJ/mol)][−(242.82 kJ/mol)(11,123 mol H₂)]=43%.

TABLE 1
Experimental conditions during integrated bioprocess run.

| Anaerobic bubble column reactor | | | | | |
|---|---|---|---|---|---|
| | | | Mode | Aerobic reactor | |
| | | | (liquid flow rate | | Mode |
| | Time | Gas composition, | to aerobic | Time | (outlet liquid |
| Phase | (hrs) | flow rate (sccm) | reactor) | (hrs) | flow rate) |
| I | 0-93 | CO/CO₂ (4/1), | Batch | 0-93 | n.s. |
| | | 1000 | | | |
| II | 94-185 | H₂/CO₂ (2/1), | Batch | 0-185 | n.s. |
| | | 1200 | | | |
| | 186-218* | H₂/CO₂ (2/1), | Continuous, | 186-218 | n.s. |
| | | 1200 | 0.5 ml/min | | |
| III | 219-312 | H₂/CO₂ (2/1), | Continuous, | 219-314** | Continuous, |
| | | 1200 | 1.5 ml/min | | 1.5 ml/min |

*~1000 ml liquid medium obtained for aerobic reactor,
n.s.: not started,
**Aerobic bioreactor inoculated with Y. lipolytica and continuous operation started.

TABLE 2
A comparison of results of single stage and integrated process.

| | | | | Product | | |
|---|---|---|---|---|---|---|
| | | | | titer-g l⁻¹ | | |
| | | | | (Lipid | | |
| | | Substrate | | content % | | Product |
| | | feeding | | of dry cell | Productivity | yield |
| Substrate | Microorganism | mode | Product | weight) | (g l⁻¹ h⁻¹) | (g g⁻¹) |
| Syngas | Moorella | Continuous | Acetic | 30 | 0.57 | NA |
| | thermoacetica | | Acid | | | |
| Acetate | Yarrowia | Fed-batch | Lipid | 46 (59%) | 0.27 | 0.16 |
| | lipolytica | | | | | |
| Syngas | Both | Continuous | Lipid | 18 (36%) | 0.19 | 0.09 |

### REFERENCES

1. Chu S, Majumdar A (2012) Opportunities and challenges for a sustainable energy future. Nature 488:294-30.

2. Tai M, Stephanopoulos G (2013) Engineering the push and pull of lipid biosynthesis in oleaginous yeast Yarrowia lipolytica for biofuel production. Metab Eng 15:1-9.

3. Blazeck J, Hill A, Liu L, Knight R, Miller J, Pan A, Otoupal P, Alper H (2014) Harnessing Yarrowia lipolytica lipogenesis to create a platform for lipid and biofuel production. Nature Comm 5:3131.

4. Liu L, Pan A, Spofford C, Nijia Zhou N, Hal Alper, H (2015) An Evolutionary Metabolic Engineering Approach for Enhancing Lipogenesis in Yarrowia lipolytica. Metab Eng 29:36-45.

5. Qiao K, Abidi SHI, Liu H, Zhang H, Chakraborty S, Watson N, Ajikumar PK, Stephanopoulos G (2015) Engineering lipid overproduction in the oleaginous yeast Yarrowia lipolytica. Metab Eng 29:56-65.

6. Shen Y, Brown R, Wen Z (2014) Enhancing mass transfer and ethanol production in syngas fermentation of Clostridium carboxidivorans P7 through a monolithic biofilm reactor. Applied Energy 136:68-76.

7. Hu P, Rismani-Yazdi H, Stephanopoulos G (2013) Anaerobic CO2 fixation by acetogenic bacterium Moorella thermoacetica, AICHE 59:3176-3183.

8. Schuhmann, K, Muller, V (2014). Autotrophy at the thermodynamic limit of life: a model for energy conservation in acetogenic bacteria. Nat Rev Microbiol 12:809-821.

9. Daniel S L, Hsu T, Dean S I, Drake H L (1990). Characterization of the H₂ and CO-dependent chemolithotrophic potentials of the acetogens Clostridium thermoaceticum and Acetogenium kivui. J Bacteriol 172:4464-71.

10. Fontanille P, et al. (2012) Bioconversion of volatile fatty acids into lipids by the oleaginous yeast Yarrowia lipolytica. Biores Technol 114:443-449.

11. Knothe G (2009) Improving biodiesel fuel properties by modifying fatty ester composition. Energy Environ Sci 2: 759-766.

12. Abubackar H N, Veiga M C, Kennes C (2011) Biological conversion of carbon monoxide: rich syngas or waste gases to bioethanol. Biofuel Bioprod Bioref 5:93-114.

13. Tanner R S, Miller L M, Yang D (1993) Clostridium ljungdahlii sp. nov., an acetogenic species in clostridial rRNA homology group I. Int J Syst Bacteriol 43:232-6.

14. William E B, Schoberth S, Tanner R S, Wolfe R S (1997) Acetobacterium, a New Genus of Hydrogen-Oxidizing, Carbon Dioxide-Reducing, Anaerobic Bacteria. Int J Syst Evol Microbiol 27: 355-361.

15. Papoutsakis E T (2015) Re-assessing the progress in the production of advanced biofuels in the current competitive environment and beyond: what are the successes and where progress eludes us and why. Ind Eng Chem Res 10.1021/acs.iecr.5b01695.

16. Hill J, Nelson E, Tilman D, Polasky S, Tiffany D (2006) Environmental, economic, and energetic costs and benefits of biodiesel and ethanol biofuels. Proc Natl Acad Sci USA 103: 11206-11210.

17. Köpke M, Mihalcea C, Bromley J C, Simpson S D (2011) Fermentative production of ethanol from carbon monoxide. Curr Opin Biotechnol 22: 320-325.

18. Wilkins M R, Atiyeh H K (2011) Microbial production of ethanol from carbon monoxide. Curr Opin Biotechnol 22: 326-330.

19. Nybo S E, Khan N E, Woolston B M, Curtis W R (2015) Metabolic engineering in chemolithoautotrophic hosts for the production of fuels and chemicals. Metab Eng 30:105-120.

20. Chakraborty S (2013). Exploring volatile fatty acids (VFAs) as a novel substrate for microbial oil production. Thesis. Massachusetts Institute of Technology.

21. Von Stockar U, and Liu J (1999) Does microbial life always feed on negative entropy? Thermodynamic analysis of microbial growth. Biochemi Biophys ACTA 1412:191-211.

22. Fei Q, et al. (2011) The effect of volatile fatty acids as sole carbon source on lipid accumulation by Cryptococcus albidus for biodiesel production. Biores Technol 102:2695-2701.

23. Seifritz C, Fröstl J M, Drake H L, Daniel S L (2002) Influence of nitrate on oxalate- and glyoxylate-dependent growth and acetogenesis by Moorella thermoacetica. Archives Microbiol 178:457-64.

24. Damude H G, et al. (2011) High Eicosapentaenoic Acid Producing Strains of Yarrowia Lipolytica. E. I. Du Pont De Nemours And Company, assignee. U.S. Pat. No. 7,932,077 B2. 26 Apr. 2011.

25. Griffiths J S, Van Hille R P, Harrison S T L (2010) Selection of direct transesterification as the preferred method for assay of fatty acid content of microalgae. Lipids 11:1053-1060.

26. Xu D, Lewis R S (2012) Syngas fermentation to biofuels: Effects of ammonia impurity raw syngas on hydrogenase activity. Biomass Bioenergy 45:303-310.

27. Wang S, Huang H, Thauer R K (2013) A reversible electron-bifurcating ferredoxin- and NAD-dependent [FeFe]-hydrogenase (HydABC) in Moorella thermoacetica. J Bacteriol 195:1267-1275.

28. Berg J M, Tymoczko J L, Stryer L (2006) Biochemistry (Freeman, N.Y.), 6th Ed. 29. van Milgen J (2002) Modeling biochemical aspects of energy metabolism in mammals. J Nutr 132(10):3195-3202.

30. Shuler M L, Kargi F (2001) Bioprocess Engineering: Basic Concepts (Prentice Hall, N.J.), 2nd Ed.

31. Verduyn C (1991) Physiology of yeasts in relation to biomass yields. Antonie van Leeuwenhoek 60(3-4):325-353.

All publications, patents and sequence database entries mentioned herein, including those items listed below, are hereby incorporated by reference for the teachings referenced herein as if each individual publication or patent was specifically and individually indicated to be incorporated by reference. In case of conflict, the present application, including any definitions herein, will control.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

Articles such as “a,” “an,” and “the,” as used herein, may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim or another portion of the description. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A method of converting a gaseous substrate comprising CO₂ into a lipid, comprising
(a) culturing a first organism in the presence of the gaseous substrate, under conditions suitable for the first organism to reduce the CO₂ in the presence of one or more reducing agents, optionally H₂ or CO, wherein the organism synthesizes one or more volatile fatty acid(s) by reduction of the CO₂, and
(b) culturing a second organism in the presence of the volatile fatty acid(s) produced in (a) under conditions suitable for the organism to convert the volatile fatty acid(s) into lipid.

2. The method according to claim 1, wherein (a) and (b) are integrated comprising a continuous processing scheme producing lipid from gas comprising CO₂.

3. The method according to claim 1 or claim 2, wherein the gaseous substrate comprises H₂, CO, CO₂ or a mixture thereof.

4. The method according to any one of claims 1-3, wherein the gaseous substrate comprises a synthesis gas (syngas).

5. The method according to any one of claims 1-4, wherein the volatile fatty acid(s) is acetic acid.

6. The method according to any one of claims 1-5, wherein the first organism is selected from the group consisting of Moorella thermoacetica, Clostridium ljungdahlii, Clostridium carboxidivorans P7T, Clostridium ragsdalei, Alkalibaculum bacchi, C. autoethanogenum, Clostridium drakei, and Butyribacterium methylotrophicum.

7. The method according to any one of claims 1-6, wherein the first organism captures carbon sourced from carbon dioxide as acetyl-CoA with a rate that is at least 1 g acetic acid/L-hr.

8. The method according to any one of claims 1-7, wherein the first organism captures carbon sourced from carbon dioxide as acetyl-CoA with an efficiency that is at least 92%.

9. The method according to any one of claims 1-8, wherein the first organism has an optimal growth temperature (Tₒₚₜ) greater than 40° C.

10. The method according to any one of claims 1-9, wherein the first organism is Moorella thermoacetica.

11. The method according to any one of claims 1-10, wherein the second organism is Yarrowia lipolytica.

12. The method according to claim 11, wherein the Yarrowia lipolytica is genetically modified to enhance lipid production.

13. The method according to claim 12, wherein the genetic modification comprises upregulation of one or more genes whose products push carbon flux into the pathway leading to lipid synthesis.

14. The method according to claim 12, wherein the genetic modification comprises upregulation of one or more genes whose products pull carbon flux through the pathway leading to lipid synthesis.

15. The method according to claim 12, wherein the genetic modification comprises upregulation of one or more genes whose products push carbon flux into the pathway leading to lipid synthesis and upregulation of one or more genes whose products are responsible for pulling carbon flux through the pathway leading to lipid synthesis.

16. The method according to any one of claims 1-15, wherein the second organism comprises at least one genetic modification providing increased expression of acetyl-coenzyme A carboxylase and/or comprises at least one genetic modification providing increased expression of diacylglycerol acyltransferase.

17. The method according to any one of claims 1-16, wherein the lipid comprises triacylglyceride.

18. The method according to claim 17, wherein the triacylglyceride comprises fatty acid groups that are less than 50% saturated fatty acid groups.

19. The method according to claim 18, wherein the triacylglyceride comprises fatty acid groups that are more than 50% oleate (C18.1), linolinate (C18.2) and palmitoleate (C16.1).

20. The method according to any one of claims 1-19, wherein (b) achieves a lipid titer of at least 15 grams per liter.

21. The method according to any one of claims 1-20, wherein (b) achieves a lipid content of at least 30%.

22. The method according to any one of claims 1-21, wherein carbon dioxide produced by the process in (b) is used as a feed gas for the process in (a).

23. The method according to any one of claims 1-22, wherein non-lipid biomass generated by the process in (b) is used as a cell culture media component for use in processes (a) and (b).

24. The method according to any one of claims 1-23, wherein the energetic efficiency of acetate to lipid conversion in the integrated process is greater than 50%, measured as the conversion of energy in the hydrogen feed to lipid produced, wherein the lipid produced is modeled as tripalmitin.

25. The method according to claim 24, wherein the energetic efficiency is greater than 75%.
